(19) United States
(12) Patent Application Publication
(10) Pub. No.: US 2026/0174491 A1
(43) Pub. Date: Jun. 25, 2026

(54) ELECTROSURGICAL SYSTEM INCLUDING PUNCTURE DETERMINATION

(71) Applicant: Boston Scientific Scimed Inc., Maple Grove, MN (US)

(72) Inventors: STEVEN KINIO, Pickering (CA); Christian Balkovec, Cambridge (CA); Deep Patel, Waterdown (CA); Eduardo Moriyama, Richmond (CA); Gareth Davies, Toronto (CA); Robert Harrison, Norval (CA); John Paul Urbanski, Toronto (CA)

(21) Appl. No.: 19/540,314

(22) Filed: Feb. 13, 2026

Related U.S. Application Data
(63) Continuation of application No. 19/257,328, filed on Jul. 1, 2025.
(60) Provisional application No. 63/667,044, filed on Jul. 2, 2024.

(51) Int. Cl. A61B 18/12; A61B 18/00; A61B 18/14; A61B 18/16

(52) U.S. Cl. CPC A61B 18/1206; A61B 18/16; A61B 2018/00642; A61B 2018/00666; A61B 2018/00708; A61B 2018/00827; A61B 2018/00875; A61B 2018/00892; A61B 2018/00904; A61B 2018/144

(57) ABSTRACT

An electrosurgical generator is disclosed. The electrosurgical generator includes device terminals including an active terminal and a return terminal and a controller. The controller generates a radiofrequency (RF) energy signal for delivery to the active terminal, measures an electrical impedance between device terminals and a phase angle of the RF energy signal while the RF energy signal is applied to the active terminal, determines at least one of a sustained change in electrical impedance, a change in electrical impedance via an impedance slope, and a change in phase angle change, and generates an indication of tissue crossing based on the determined change of the electrical impedance or the phase angle.

### CROSS REFERENCE TO RELATED APPLICATION

This application is a continuation of U.S. patent application Ser. No. 19/257,328 entitled “ELECTROSURGICAL SYSTEM INCLUDING PUNCTURE DETERMINATION,” filed Jul. 1, 2025, which claims priority to U.S. Provisional Patent Application No. 63/667,044 entitled “ELECTROSURGICAL SYSTEM INCLUDING PUNCTURE DETERMINATION,” filed Jul. 2, 2024, which is hereby incorporated by reference in its entirety.

### TECHNICAL FIELD

The present disclosure relates to medical devices and systems for use in percutaneous or interventional procedures including surgery such as electrophysiology procedures. More specifically, this disclosure relates to electrosurgical units, such as radiofrequency (RF) generators, electrosurgical systems, and methods, that provide for measurements of electrical characteristics to crossing members used with RF generators.

### BACKGROUND

Catheters are often used to provide general access into a patient's body using minimally invasive techniques. In some examples, a catheter can be used to create a channel through a region of the body. For instance, punctures in tissues can provide access for medical tools used in various medical interventions. In one example, a pericardium layer of a patient can be punctured to provide for epicardial access, such as to create an access point to insert tools for epicardial ablation. In another example, electrosurgical devices are applied to remove accumulation of atheromatous material on the inner walls of vascular lumens, which results in atherosclerosis. In one technique, an electrosurgical device is applied to puncture through the vascular occlusion without affecting the vessel walls. Another example is a transseptal puncture in a cardiac procedure. The left atrium is a difficult cardiac chamber to reach percutaneously. Although the left atrium can be reached via the left ventricle and mitral valve, the catheter is manipulated through two U-turns, which can be cumbersome. the transseptal puncture is a technique of creating a small passage through the atrial septum, or wall in the heart between the left and right atrium, through which a catheter can be fed. The atrial septum is punctured and dilated via tools. The transseptal puncture permits a direct route to the left atrium via the intra-atrial septum and systematic venous system. Increasing larger and complex medical devices can be passed into the right atrium.

Punctures, such as transseptal punctures, can be performed with the aid of electrosurgical crossing devices, such as transseptal guidewires, having electrodes energized with a suitable power source such as an electrically coupled electrosurgical generator to provide the source of RF energy in a manner like other electrosurgical instruments. Typical electrosurgical instruments including electrosurgical crossing devices apply an electrical potential difference or a voltage difference between an active electrode and a return electrode on a patient's grounded body in a monopolar arrangement of the device or between an active electrode and a return electrode on the device in bipolar arrangement to deliver the RF energy to the area where tissue is to be affected. Electrosurgical crossing devices pass RF energy through tissue between the electrodes to puncture tissue with plasma formed on the energized electrode. Tissue that contacts the plasma experiences a rapid vaporization of cellular fluid to produce a puncture effect. Electrical energy can be applied to the electrodes either as a train of high frequency pulses or as a continuous signal typically in the RF range to perform the puncturing techniques.

### SUMMARY

In Example 1, an electrosurgical generator comprising: a plurality of device terminals including an active terminal and a return terminal; and a controller configured to generate a radiofrequency (RF) energy signal for delivery to the active terminal, measure an electrical impedance between the plurality of device terminals and a phase angle of the RF energy signal while the RF energy signal is applied to the active terminal, determine at least one of: a sustained change in electrical impedance, a change in electrical impedance via an impedance slope, and a change in phase angle change, and generate an indication of tissue crossing based on the determined change of the electrical impedance or the phase angle.

In Example 2, the electrosurgical generator of Example 1, wherein the controller is configured to terminate the energy signal as the indication of tissue crossing based on the determined changes of the electrical impedance and the phase angle.

In Example 3, the electrosurgical generator of any of Examples 1 and 2, wherein the change in phase angle includes a change from a relatively resistive phase angle to a relatively capacitive phase angle.

In Example 4, the electrosurgical generator of Example 3, wherein the change of the phase angle is based on a preselected phase angle threshold between zero degrees and negative ninety degrees.

In Example 5, the electrosurgical generator of Examples 4, wherein the selected phase angle threshold includes a resistive phase angle threshold and a capacitive phase angle threshold, wherein the resistive phase angle threshold is greater than the capacitive phase angle threshold.

In Example 6, the electrosurgical generator of any of Examples 1-5, wherein the change in electrical impedance is inferred from a change in current applied to the active terminal.

In Example 7, the electrosurgical generator of Example 6, wherein the change in electrical impedance is determined from the current exceeding a current threshold.

In Example 8, the electrosurgical generator of any of Examples 1-7, wherein the indication of tissue crossing is generated based on the sustained change in electrical impedance or on a sustained change in phase angle, wherein the sustained change is measured over a period of time.

In Example 9, the electrosurgical generator of any of Examples 1-7, wherein the indication of tissue crossing is generated based on the sustained change in electrical impedance or on a sustained change in phase angle, wherein the sustained change is measured over a plurality of successive readings of the impedance or the phase angle.

In Example 10, the electrosurgical generator of Example 1, wherein the change in the phase angle is based on a selected phase angle threshold and a selected phase angle slope.

In Example 11, the electrosurgical generator of Example 10, wherein the change in the phase angle includes a determination that the monitored phase angle is more resistive than the selected phase angle threshold.

In Example 12, the electrosurgical generator of any of Examples 10 and 11, wherein the change in phase angle further comprises a subsequent phase angle slope.

In Example 13, the electrosurgical generator of any of Examples 1-12, wherein the puncture system further includes a crossing device having an electrode, the crossing device electrically coupled to the active terminal.

In Example 14, the electrosurgical generator of Example 1, wherein the controller is configured to facilitate presentation of the electrical impedance and the phase angle on a display device as the indication of tissue crossing based on the determined changes of the electrical impedance and the phase angle.

In Example 15, the electrosurgical generator of any of Examples 14, wherein the controller is configured to facilitate presentation of a graph of the electrical impedance and the phase angle as a function of time on a display device as the indication of tissue crossing based on the determined changes of the electrical impedance and the phase angle.

In Example 16, an electrosurgical generator comprising: a plurality of device terminals including an active terminal and a return terminal; and a controller configured to generate a radiofrequency (RF) energy signal for delivery to the active terminal, measure an electrical impedance between the plurality of device terminals and a phase angle of the RF energy signal while the RF energy signal is applied to the active terminal, determine at least one of: a sustained change in electrical impedance, a change in electrical impedance via an impedance slope, and a change in phase angle change, and generate an indication of tissue crossing based on the determined change of the electrical impedance or the phase angle.

In Example 17, the electrosurgical generator of Example 16, wherein the change in phase angle includes a change from a relatively resistive phase angle to a relatively capacitive phase angle.

In Example 18, the electrosurgical generator of Example 17, wherein the change in the phase angle is based on a preselected phase angle threshold.

In Example 19, the electrosurgical generator of Example 18, wherein the preselected phase angle is between zero degrees and negative ninety degrees.

In Example 20, the electrosurgical generator of Example 18, wherein the selected phase angle threshold includes a resistive phase angle threshold and a capacitive phase angle threshold, wherein the resistive phase angle threshold is greater than the capacitive phase angle threshold.

In Example 21, the electrosurgical generator of Example 16, wherein the change in electrical impedance is inferred from a change in current applied to the active terminal.

In Example 22, the electrosurgical generator of Example 21, wherein the change in electrical impedance is determined from the current exceeding a current threshold.

In Example 23, the electrosurgical generator of Example 16, wherein the change in electrical impedance is based on a voltage and a current applied to the active terminal.

In Example 24, the electrosurgical generator of Example 16, wherein the indication of tissue crossing is generated based on the sustained change in electrical impedance or on a sustained change in phase angle, wherein the sustained change is measured over a period of time.

In Example 25, the electrosurgical generator of Example 16, wherein the indication of tissue crossing is generated based on the sustained change in electrical impedance or on a sustained change in phase angle, wherein the sustained change is measured over a plurality of successive readings of the impedance or the phase angle.

In Example 26, the electrosurgical generator of Example 16 wherein the change in the phase angle is based on a selected phase angle threshold and a selected phase angle slope.

In Example 27, the electrosurgical generator of Example 26, wherein the change in the phase angle includes a determination that the monitored phase angle is more resistive than the selected phase angle threshold.

In Example 28, the electrosurgical generator of Example 26, wherein the change in phase angle further comprises a subsequent phase angle slope and the subsequent phase angle slope includes a positive slope.

In Example 29, the electrosurgical generator of Example 28, wherein the controller is configured to terminate the energy signal as the indication of tissue crossing based on the determined changes of the electrical impedance and the phase angle.

In Example 30, an electrosurgical tissue puncture system comprising: a plurality of device terminals including an active terminal and a return terminal; a crossing device having a puncture electrode, the crossing device electrically coupled to the active terminal; a ground pad dispersive electrode electrically coupled to the return terminal; and a controller configured to generate a radiofrequency (RF) energy signal for delivery to the active terminal, measure an electrical impedance between the plurality of device terminals and a phase angle of the RF energy signal while the RF energy signal is applied to the active terminal, determine an impedance change and a phase angle change, and generate an indication of tissue crossing based on the determined changes of the electrical impedance and the phase angle.

In Example 31, the electrosurgical tissue puncture system of Example 30, wherein the crossing device includes a transseptal guidewire.

In Example 32, a method for use with an electrosurgical generator having a plurality of device terminals including an active terminal and a return terminal, the method comprising: generating a radiofrequency (RF) energy signal for delivery to the puncture electrode of the crossing device; measuring an electrical impedance between the plurality of device terminals and a phase angle of the RF energy signal while the RF energy signal is applied to the active terminal; determining an impedance change and a phase angle change; and terminating the RF energy signal based on the determined changes of the electrical impedance and the phase angle.

In Example 33, the method of Example 32, wherein determining the change in phase angle includes determining a change from a relatively resistive phase angle to a relatively capacitive phase angle as determined from a preselected phase angle threshold.

In Example 34, the method of Example 33, wherein determining the change in phase angle is further based on a selected phase angle slope.

In Example 35, the method of Example 32, wherein terminating the RF energy signal wherein the energy signal is based on determining a sustained change of the electrical impedance or the phase angle.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary electrosurgical system for performing an electrosurgical puncture, such as a transseptal puncture.

FIG. 2 is a schematic diagram illustrating a radiofrequency generator for use in the electrosurgical system of FIG. 1.

FIG. 3 is a graph having plots of impedance magnitude and phase angle with respect to time in a transseptal puncture.

FIG. 4 is a schematic diagram illustrating various stages of the transseptal puncture corresponding with the graph of FIG. 3.

FIG. 5 is a flow diagram illustrating a method for terminating an energy signal applied in the transseptal puncture of FIG. 4.

FIG. 5A is a flow diagram illustrating a method that is an embodiment of the method of FIG. 5.

FIG. 6 is a graph illustrating plots of current and phase angle with respect to time corresponding with the method of FIG. 5.

FIG. 7 is a flow diagram illustrating a method that is an embodiment of the method of FIG. 5.

FIG. 7A is a flow diagram illustrating a method that is an embodiment of the method of FIG. 7.

FIG. 8 is a block diagram illustrating a method that is an embodiment of the method of FIG. 5.

FIG. 9 is a graph illustrating plots of current and phase angle with respect to time corresponding with the method of FIG. 8.

FIG. 10 is another graph illustrating plots of current and phase angle with respect to time corresponding with the method of FIG. 8.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

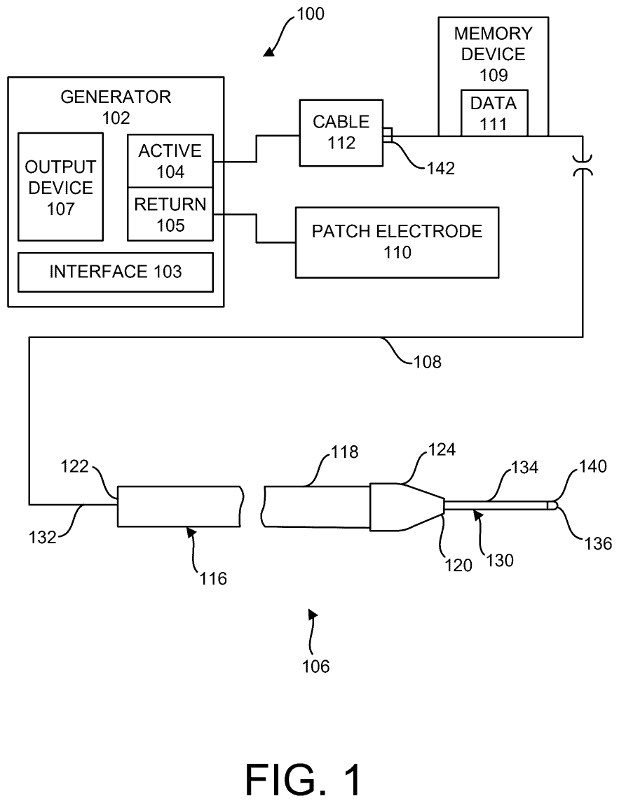

FIG. 1 illustrates an embodiment of an electrosurgical system 100 to facilitate vascular access to a heart and provide catheter positioning within cardiac anatomy. The embodiment of the medical system 100 includes an electrosurgical unit, such as an electrosurgical generator 102 and an electrosurgical device, such as an electrosurgical puncture device 106. In one example, the electrosurgical puncture device 106 includes an electrosurgical transseptal guidewire 108. In the illustration, the electrosurgical transseptal guidewire 108 is electrically coupled to the electrosurgical generator 102 via cable 112, such as to an active terminal 104 on the electrosurgical generator 102. In embodiments, the electrosurgical generator can be coupled to other electrosurgical devices or other electrosurgical puncture devices including electrosurgical transseptal puncture devices. The electrosurgical generator 102 is configured to provide a source of energy, such as radiofrequency (RF) energy to the electrosurgical transseptal guidewire 108 via the cable 112. In some embodiments, the system 100 includes a ground pad electrode, or indifferent (dispersive) patch electrode 110 electrically coupled to the electrosurgical generator 102, such as to a return terminal 105 on the electrosurgical generator 102, for use with the electrosurgical transseptal guidewire 108 in a monopolar configuration. A In some embodiments, the electrosurgical transseptal guidewire is implemented in a bipolar configuration using a pair of electrodes on the guidewire and without a separate patch electrode.

In one embodiment, the electrosurgical system is an electrosurgical tissue puncture system configured to puncture biological tissue in a patient, such as during an electrophysiological procedure. The electrosurgical generator 102 is configured to provide the source of RF energy to the electrosurgical transseptal guidewire 108 for a puncture operation with the electrosurgical device 106. The electrosurgical generator 102 includes an interface 103 including a set of user accessible controls, device connectors such as active terminal 104 and a return terminal 105, and an output device 107 such as a display device, speakers, and lights. During a monopolar puncture operation of electrosurgical generator 102, a first electrode, often referred to as the active electrode, is provided with the electrosurgical device 106 in general and with the transseptal guidewire 108 in the illustration while a second electrode, such as patch electrode 110, is typically located on the back, buttocks, upper leg, or other suitable anatomical location of the patient during surgery. In such a configuration, the patch electrode 110 is often referred to as a patient return electrode. The active electrode is electrically coupled to the active terminal 104 and the patch electrode 110 is electrically coupled to the return terminal 105. (In some embodiments, the transseptal guidewire includes multiple active electrodes electrically coupled to the active terminal 104 or the return terminal is coupled to multiple patch electrodes.) An electrical circuit of RF energy is formed between the active electrode and the patch electrode 110 through the patient, which is used to puncture tissue at the active electrode. For example, RF energy for a puncture function in a monopolar mode may be provided at a relatively low voltage and a continuous current (100% on, or 100% duty cycle). At a power setting of 50 Watts for puncturing (although instantaneous power may be higher), voltage can range from approximately 164 to 400 volts root mean square (RMS). The electrosurgical generator 102 can include a plurality of functions and provide a programmed and custom settings via an interface and be couplable to a suite of electrosurgical devices in addition to the transeptal guidewire 108. In one example, the electrosurgical generator 102 provides RF energy to the active electrode as an alternating current having a frequency in the range of 100 kHz to 10 MHz. Typically, this energy is applied in the form of a continuous sinusoidal energy signal. In some embodiments, the energy is applied in bursts of pulses. The individual pulses in each burst of a pulsed energy signal typically each have a duration of 300 milliseconds with an interval between pulses of 700 milliseconds but can vary such as based on parameters of the connected electrosurgical device 106. The actual pulses are often sinusoidal or square waves and bi-phasic, that is alternating positive and negative amplitudes.

In one example, the electrosurgical generator 102 provides the power to the electrosurgical puncture device 106, but the actual power level delivered to the electrosurgical puncture device 106 can be selected via controls on the electrosurgical puncture device 106 rather than controls on the electrosurgical generator 102. In another example, the electrosurgical generator 102 can be programmed to provide power levels within a selected range of power, and the electrosurgical puncture device 106 is used to select an output power level within the preprogrammed range. For instance, the electrosurgical generator 102 can be programmed to provide monopolar energy for a puncture function in a first range of power settings as well as voltage-based controls to target a specific voltage. The electrosurgical generator 102 can be programmed to provide monopolar energy for another function in a second range of power or voltage settings, which second range may be the same as, different than, or overlap the first range. In some embodiments, the user may then select the function and adjust the power or voltage setting within the range using controls on the electrosurgical puncture device 106 rather than using controls on the electrosurgical generator 102.

In one embodiment, the electrosurgical generator 102 can program and select particular controls, or ranges of controls, based on the particular configuration of the electrosurgical transeptal guidewire 108. The transseptal guidewire of the embodiment includes a memory device 109 (non-transitory memory) storing a set of parameters 111 associated with the transseptal guidewire 108. The electrosurgical generator 102 is configured to read the parameters to program the controls to be suited for the associated transseptal crossing device. The memory device 109 can store the parameters 111 in various memory segments having lookup tables or other data structures to provide data to be loaded into a memory device in the electrosurgical generator 102 and read by a controller of the electrosurgical generator to affect operation. Example parameters 111 can include model number of the transseptal guidewire 108, acceptable power levels signals applied to the transseptal device 108, whether the transseptal device is configured for single use or multiple uses, as well as other parameters. In some embodiments, the electrosurgical generator 102 can be programmed to write to memory segments on the memory device 109 as well as read the memory device 109.

The illustrated electrosurgical puncture device 106 includes the electrosurgical transseptal guidewire 108 and a delivery component 116. While embodiments of the disclosure are described with reference to punctures in tissue with a transseptal guidewire for illustration, the features of the disclosure can be used with other electrosurgical devices including other transseptal surgical devices such as needle-based platforms. The delivery component 116 includes an elongated shaft 118 having a shaft distal tip 120. The elongated shaft 118 defines a longitudinally extending axial lumen 122. The electrosurgical transseptal guidewire 108 is adapted to be disposed within the lumen 122 and coupled to the RF energy source. In some embodiments, the delivery component 116 can include an elongate sheath, and the electrosurgical transseptal guidewire 108 is disposed within the sheath. In another embodiment, the delivery component 116 can include a dilator/sheath assembly, and the electrosurgical transseptal guidewire 108 is disposed within the dilator/sheath assembly. For instance, the elongated shaft 118 includes a distal tapered portion 124 with an enlargement of cross-sectional area with respect to the shaft distal tip 120. As the distal tapered portion 124 is passed through an aperture from the shaft distal tip 120, the enlargement of cross-sectional area dilates the aperture. The dilator can be configured as a straight dilator, as illustrated, or a curved dilator. The elongated shaft 118 can be made from various materials including insulative materials such as high-density polyethylene (HDPE). The shaft 118 and distal tip can include various materials such as metal hypotubes as well.

The electrosurgical transseptal guidewire 108 includes a puncture wire shaft 130 with a puncture wire proximal portion 132 and a puncture wire distal portion 134 having a puncture wire distal tip 136. The puncture wire distal tip 136 includes a puncture electrode 140 adapted to deliver the RF energy. The puncture electrode 140 is configured as the active electrode. The puncture wire proximal portion 132 includes an end connector 142 configured to electrically couple to cable 112 and receive an RF signal from the electrosurgical generator 102. In one example, the electrosurgical transeptal guidewire 108 can be coupled to and uncoupled from the cable 112 depending on whether the electrosurgical transeptal guidewire 108 is used as an electrosurgical puncture device or as an exchange rail, for instance. The transseptal guidewire 108 is configured to conduct the RF signal from the proximal portion 132 along the puncture wire shaft 130 to the electrode 140. In some embodiments, the puncture wire shaft 130 is constructed from an electrically conductive material having an insulative outer coating. In some embodiments, the electrically conductive material is a flexible, shape memory material such as a nickel titanium alloy or nitinol. The exposed electrode 140 is configured to apply the RF energy, such as to puncture tissue.

In the illustrated example, the electrosurgical transseptal guidewire 108 is configured as a multifunction conductive guidewire. For instance, the transseptal guidewire 108 can be used, without exchanges, as a guidewire, a transseptal puncture device, and as an exchange rail for delivering therapy sheaths. Such embodiments provide efficiencies to medical procedures as the transseptal guidewire 108 performs multiple functions and reduces the amount of device exchanges in the medical procedure. The transseptal guidewire 108 includes a distal tip 136 extendable from the delivery component distal end 120 such that the delivery component 116 is retractable from the patient over the guidewire 108 with the guidewire distal tip 136 disposed within the heart. The transseptal guidewire 108 is sufficiently thin and flexible to access the various chambers of the heart. The electrode 140 on the puncture wire distal tip 136 is operable to deliver RF energy to puncture the atrial septum from the right atrium, and the distal portion 134 of the puncture wire shaft 130 can be advanced through the puncture. Once advanced through the puncture and sufficiently extended from within the delivery component 136, the distal portion 134 is biased to form a coil for anchoring the transseptal guidewire 108 beyond the puncture. The delivery component 116 is retractable from the patient over the transseptal guidewire 108 with the distal tip 136 still disposed within the heart. The transseptal guidewire 108 can also support the installation of therapy devices to a therapy location in the patient's heart, such as tubular members or other catheters and for advancing other devices within the heart.

In an anticipated use of the system 100, the electrosurgical device 106 is coupled to the RF generator 102, and if the electrosurgical device 106 is to be configured in a monopolar mode, the patch electrode 110 is coupled to the patient. The RF generator 102 can be set to a puncture mode, such as an energy output of approximately 50 watts. In some examples, femoral access is obtained via a conventional percutaneous needle, and the transseptal guidewire 108 is inserted into the vasculature and advanced to the superior vena cava. The shaft distal tip 120 of the delivery component 116 is advanced over the proximal portion 132 of the guidewire 108, and the distal tapered portion 124 of the delivery component shaft 118 is advanced over the guidewire 108 to the superior vena cava. Under visualization, the distal tapered portion 124 is moved from the superior vena cava to the right atrial septum and then to the fossa ovalis of the heart. Once the delivery component distal tip 120 is confirmed at the fossa ovalis, the electrode 140 of the transseptal guidewire 108 is advanced from the delivery component distal tip 120. In one example, the exposed puncture electrode 140 of the transseptal guidewire 108 is extended a few millimeters from the delivery component distal tip 120 to tent the heart tissue, and the transseptal guidewire 108 can be locked in position with respect to the delivery component 116 in some cases. Forward pressure is applied to the electrosurgical device 106 and the transseptal guidewire 108 is actuated to apply the RF energy to the electrode 140 and puncture the fossa ovalis. The RF energy punctures the fossa ovalis and creates an aperture in the fossa ovalis. In cases in which the transseptal guidewire is locked in position, the transseptal guidewire 108 is unlocked from the delivery component 116; and the transseptal guidewire 108 is extended through the aperture. In general, the transseptal guidewire 108 is extended longitudinally for several millimeters prior to the distal portion 134 curving to assume a i-tip or pigtail shape and deflecting away from the atrial septum. The transseptal guidewire 108 can be advanced into the left atrium of the heart and anchored. In the embodiment of the delivery component 116 configured as the dilator/sheath assembly, the distal tapered portion 124 of a dilator, the distal tapered portion 124 is advanced into the puncture site to expand the aperture. The delivery component 116 can be retracted from the patient over the transseptal guidewire 108, and transseptal guidewire 108 can provide support for the installation of tubular members or other catheters and for advancing other devices within the heart.

A benefit of using RF energy to puncture tissue is that the electrosurgical crossing device, such as the distal tip 136 of the transseptal guidewire 108, is generally inert and atraumatic to the patient until a clinician is ready to perform a crossing. The electrosurgical crossing device will only cut when an RF energy signal is provided from the electrosurgical generator. In some embodiments and as used for illustration in this disclosure, an energy signal is configured to puncture tissue. In other embodiments, the energy signal includes a power insufficient to puncture tissue.

Typical electrosurgical generators when activated will deliver an RF energy signal to the electrosurgical crossing device for a brief period of time. In one example, a clinician can specify an amount of time the energy signal will be applied to the crossing device. Once a clinician activates the electrosurgical generator to apply the energy signal, the crossing device will receive the energy signal for the specified amount of time, barring an error condition. For example, a clinician can select an amount of time, such as one second, for the energy signal to be delivered upon activation. Once specified, each activation of the energy signal will last one second, and the energy signal will be deactivated or terminated after one second.

Such time-based activation/deactivation of the energy signal has some disadvantages. For example, a puncture of tissue typically takes less than the full duration of the specified amount of activation time. Continued activation of the energy signal after the crossing applies excess RF energy into the blood pool, which increases thermal-withstand demands on electrical insulation adjacent to the electrode and unintended fowling of the electrode tip as surrounding blood is heated or vaporized. Additionally, many clinicians will apply the energy signal while the electrode of the crossing device is still in the delivery component rather than against the tissue in a “running start” strategy. In the running start strategy, the energy signal is applied to the electrode within the delivery component and retracted from the tissue and then the energized electrode is physically advanced to cross the target tissue. The running start strategy risks the live electrode curving back and recrossing the septum from the left atrium or contacting and traumatizing an undesired structure due to the curved tip profile.

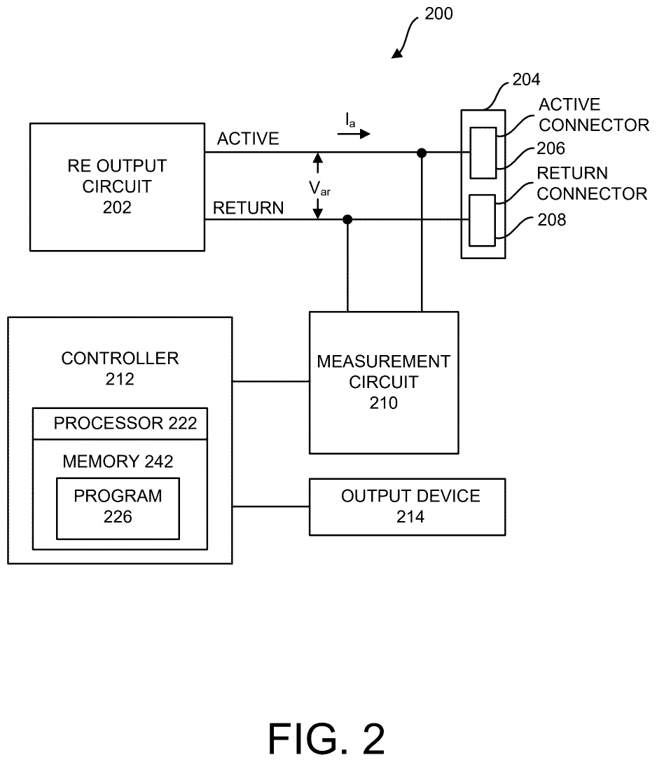

FIG. 2 illustrates an embodiment of an electrosurgical unit, such as an electrosurgical generator 200, which can correspond with electrosurgical generator 102 in system 100. The electrosurgical generator 200 is configured to couple to an electrosurgical puncture device and generate an RF energy signal; the RF energy signal is provided to the puncture device. The RF energy signal is selectively activated, such as by a clinician, to vaporize tissue. The electrosurgical generator 200 monitors electrical characteristics of the RF energy signal to determine whether tissue has been vaporized and penetrated. In embodiments, the electrosurgical generator 200 determines an electrical impedance magnitude across the active and return terminals and measures a phase angle of the RF energy signal while the energy signal is activated and applied to the active terminal. Based on measured changes of electrical impedance and electrical characteristics of the RF energy signal, the electrosurgical generator 200 infers tissue has been crossed and automatically deactivates or terminates the RF energy signal. In embodiments, the electrosurgical generator 200 is configured to automatically terminate the energy signal based on a change of the impedance magnitude and phase angle of the energy signal. In one embodiment, the change of the phase angle includes a change from a resistive phase angle to a capacitive phase angle. The automatic termination of the energy signal based on an indication of tissue crossing typically occurs before the time-based deactivation, which reduces the risk of cross back events and reduces RF energy delivered to the patient. Controls are also prepared to terminate the energy signal based on the time-based deactivation if a crossing event is not indicated prior to the time-based deactivation.

The electrosurgical generator 200 includes an RF energy output circuit 202, a plurality of device terminals, or connectors 204, including an active terminal, or active connector 206, and a return terminal, or return connector 208, a measurement circuit 210, and a controller 212. In embodiments, the RF energy output circuit 202 is configured to generate an RF energy signal. The RF energy output circuit 202 is electrically coupled to the device connectors 204 and provides an energy signal to the active connector 206. The measurement circuit 210 is electrically coupled to the RF energy output circuit 202 and the device connectors 204 to determine electrical characteristics of the energy signal, such as voltage, current, impedance, and phase angle, of the energy signal. The measurement circuit 210 is electrically coupled to the controller 212 and provides inputs representative of the electrical characteristics to the controller 212. The controller 212 is electrically coupled to the RF energy output circuit 202 and operates the RF energy output circuit 202, such as activates and terminates the energy signal. In one embodiment, a clinician can set a time limit for an amount of time apply the energy signal once the energy signal has been activated. After the time limit has expired, the controller 212 will cause the RF energy output circuit to terminate the energy signal. Typically, tissue is vaporized and a puncture is formed in the tissue very quickly and prior to expiration of the time limit set in the controller 212. In embodiments of the electrosurgical generator 200, the controller 212 causes the RF energy output circuit to terminate the energy signal based on the measured electrical characteristics indicating a likelihood that the puncture is formed, which is often prior to the expiration of the time limit.

The RF energy output circuit 202 can include a power supply to provide a direct current signal and can convert the direct current signal to an alternating current signal. The RF energy output circuit 202 is configured to generate a plurality of voltages, waveforms having various duty cycles, peak voltages, crest factors, frequencies and other suitable parameters and provide the energy signal to the active connector 206.

The device connectors 204 in embodiments include terminals, such as electrical receptacles, located on a housing of the electrosurgical generator 200 that can be mechanically coupled to a cable or an electrosurgical device and a ground pad electrode. The device connectors 204 are electrically coupled to the RF energy output circuit 202 and configured to electrically couple the puncture device and ground pad electrode to the RF energy output circuit 202. For example, the active connector 206 is suitable for electrically coupling to cable 112, which can be electrically coupled to the transeptal guidewire 108. The return connector 208 is suitable for electrically coupling to the ground pad electrode 110 when an electrosurgical device is operated in a monopolar mode or to a return electrode on the electrosurgical device when operated in a bipolar mode.

The measurement circuit 210 is electrically coupled to the device connectors 204 and RF energy output circuit 202 and is configured to determine current and voltage measurements or impedance measurements from energy signal generated by the RF energy output circuit 202 and present the current and voltage measurements to the controller 212. The measurement circuit 210 can include circuit elements or paths electrically coupled to the RF energy output circuit 202 or at least some of the output connectors 204 including the active connector 206 and return connector 208 and is configured to provide a signal representative of the active and return voltages and active current. The circuit elements can include current probes to measure currents of interest. In some embodiments, the measurement circuit 210 generates a measurement of the current of the energy signal from the active connector 206, and in other embodiments, the measurement circuit 210 generates a measurement of the of the current of the energy signal from the return connector 208. In one embodiment, the measurement circuit 210 includes an analog to digital converter coupled to the circuit elements and the controller 212 to provide digital signals to the controller 212.

In embodiments, the controller 212 is implemented with any combination of hardware and programming to receive inputs from the measurement circuit 210 and operate the RF energy output circuit 202. In one embodiment, the controller 212 incudes a processor 222 operably coupled to a memory device 224 (a tangible storage medium). The memory device 224 can store processor-executable instructions configured to control the processor 222, such as a program 226. Examples of a memory device 224 can include a non-volatile memory device such as a read only memory (ROM), electronically programmable read only memory (EPROM), flash memory, non-volatile random-access memory (NRAM) or other memory device, and a volatile memory device such as random-access memory (RAM) or other memory device. Memory device 224 can include various combinations of one or both of non-volatile memory devices and volatile memory devices. The processor 222 includes an output port that allows the processor 222 to control the RF energy output circuit 202 according to a selected scheme.

In other embodiments, the functionalities of controller 212 are at least partially implemented in the form of electronic circuitry. Examples of electronic circuitry include integrated circuits including ASICs and programmable logic devices, such as field programmable gate arrays. A field programmable gate array is a type of integrated circuit that can be programmed or reprogrammed after manufacture and include programable logic blocks and interconnects that are configured to perform various digital functions. The logic blocks can be configured to perform combinational functions or as logic gates. Logic blocks can also include memory elements, such as flip-flops or more complete memory devices including volatile and non-volatile memory aspects that can include look up tables. Functions can be defined via a hardware description language in an electronic design automation tool to create a binary file to configure the electronic circuitry.

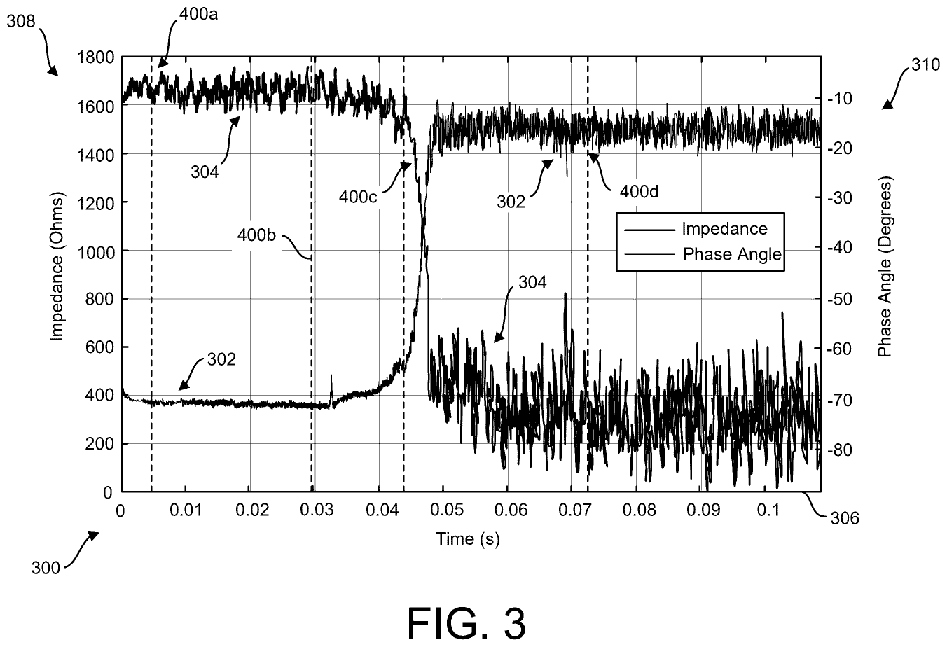

FIG. 3 illustrates a graph 300 including a plot of electrical impedance magnitude 302 superimposed with a plot of phase angle 304 of an energy signal as measured at the electrosurgical generator 200 with respect to time as a transseptal guidewire is applied to form a puncture in target tissue in an example procedure. A timeline, or time since activation of the energy signal in the example procedure in seconds 306 is presented along the x-axis. Electrical impedance magnitude in Ohms 308 is presented along the left-side y-axis. Phase angle in degrees 310 is presented along the right-side y-axis. Time 306 of the procedure ranges from zero seconds to over 0.1 seconds. Impedance magnitude on the graph along the left-side y-axis ranges from zero Ohms to 1800 Ohms. Phase angle 310 on the graph along the right-side y-axis ranges from negative ninety degrees to zero degrees. The graph ranges in this disclosure are for illustration, and other ranges can be used, such as impedance magnitudes greater than or less than 1800 Ohms. In some embodiments, the impedance magnitude may be as high as about 3500 Ohms. Also, phase angles can include ranges from less than or greater than negative ninety degrees to greater than or less than zero degrees. Resistive phase angles are at approximately zero degrees and capacitive phase angles are at approximately negative ninety degrees. For instance, resistive phase angles are greater than negative forty-five degrees and capacitive phase angles are less than negative forty-five degrees. In some examples, circuitry in the generator 200 can induce a positive phase angle, such as approximately three degrees, in an otherwise resistive electrical load applied at the active and return electrodes.

Typical transseptal punctures exhibit a distinctive electrical event as visualized in graph 300. As illustrated in plots 302, 304, a relatively large change in both electrical impedance magnitude and phase angle are observed during a septal crossing. Based on the plot of the phase angle 304, the RF energy signal delivery appears to change from a resistive delivery to a capacitive delivery, with significantly higher impedance during the capacitive delivery stages. The timescale on the events can vary, but typically the change from resistive delivery to capacitive delivery occurs in the first fifty milliseconds to one hundred milliseconds of an application of an RF energy signal to the septum.

The inventors hypothesized that the distinctive electrical event indicated a transseptal crossing. To test the hypothesis, a high-speed camera system was developed in conjunction with a blood-analog liquid media and a porcine model to observe the effects during the plots 302, 304. Upon observation, the change in the impedance plot 302 from a relatively low impedance magnitude to a relatively high impedance magnitude and the change in the phase angle plot 304 from a relatively resistive phase angle to a relatively capacitive phase angle occurred at the time of transseptal crossing. The distinctive changes in the plots 302, 304 is due to the design of the electrodes used in the test of the hypothesis, that is, the design allows the heated electrode to capture vapor generated by the electrode in the liquid medium. While the electrode is in contact with the tissue, the vapor barrier is displaced from the electrode at contact, which allows the relatively highly resistive delivery of the energy signal to the tissue. Once the tissue has been vaporized, the vapor layer will encapsulate the heated electrode, which provides an electrically insulative gaseous layer between the electrode and the conductive liquid medium, such as blood, that results in a relatively highly capacitive delivery of the energy signal. Real impedance rises at the crossing, and the RF energy now flows through alternate parasitic capacitive pathways.

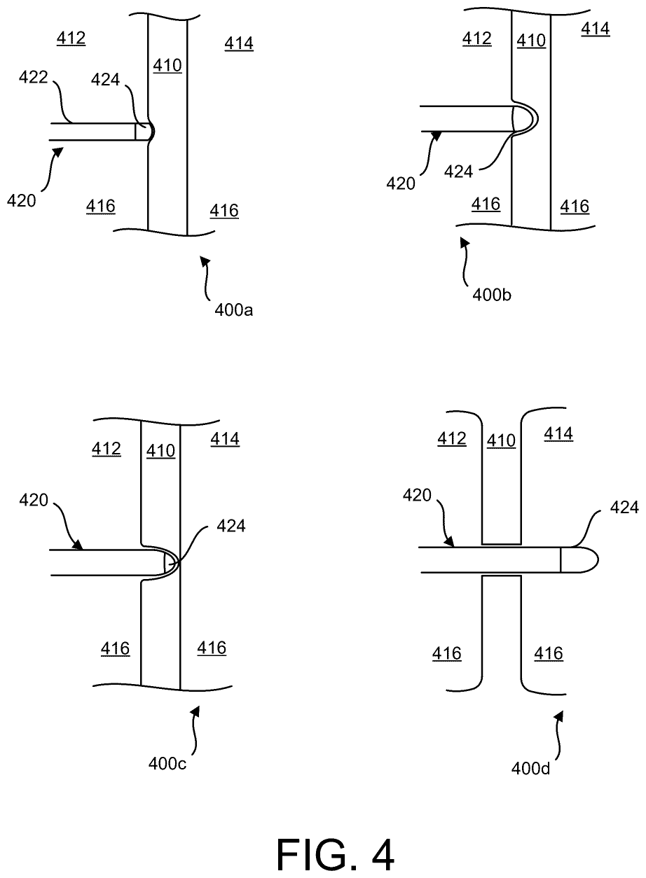

FIG. 4 illustrates schematic views of various stages of the procedure 400 overtime such as a first stage 400a, second stage 400b, third stage 400c, and fourth stage 400d, which are correlated along the timeline on the graph 300 of FIG. 3 for illustration. The stages 400 illustrate target tissue, such as an atrial septum 410 separating a right atrium 412 and a left atrium 414, in a liquid, such as blood 416. A puncture device 420, such as the transseptal guidewire 108 or another suitable electrosurgical puncture device, includes a shaft 422 and electrode 424. The electrode receives an energy signal, and electrical characteristics of the energy signal are plotted on graph 300 of FIG. 3.

Early in the illustrated timeline, as illustrated in the first stage 400a, the electrode 424 is urged against the target tissue 410 from the right atrium 412, and an energy signal is applied to the electrode 424. The electrode 424 is rapidly heated and electrical current from the energy signal flows into the target tissue 410. The first stage 400a corresponds with a relatively low measured impedance on the plot of impedance 302 and a relatively resistive measured phase angle on plot of phase angle 304.

Subsequently, as illustrated in the second stage 400b, the electrode 424 begins to vaporize tissue 410 with the energy signal. The electrode 424 is at a relatively stable temperature and electrical current from the energy signal flows into the target tissue 410. The second stage 400b corresponds with a relatively low measured impedance on the plot of impedance 302 and a relatively resistive measured phase angle on plot of phase angle 304.

As the puncture device 420 is about to penetrate the target tissue 410, illustrated in the third stage 400c, substantial changes in the plots 302, 304 occur. The electrode 424 has vaporized the target tissue 410 and begins to contact the blood 416 of the left atrium 414. Current from the energy signal applied to the electrode 420 flows into the target tissue 410 and now begins to flow into the blood 416. The energy signal maintains relatively stable heating to the electrode 424. The third stage 400c corresponds with transition to a relatively higher measured impedance on the plot of impedance 302 and a concurrent transition to a relatively more capacitive measured phase angel on the plot of phase angle 304.

As the puncture device 420 fully penetrates target tissue 410, as illustrated in the fourth stage 400d, the electrode 424 is immersed in the blood 416 of the left atrium 414, which effects the plots 302, 304. The electrode 424 has vaporized the target tissue to form an aperture 450. As the energy signal remains applied to the electrode 424, the electrode 424 heats the surrounding blood 416 and bubbles 460 begin to form on and adhere to the electrode 424. The bubbles 460 serve to provide an electrically insulative effect on the electrode 424. In some examples, the bubbles 460 coalesce and form an electrically insulative gaseous layer 470 encapsulating the entire electrode 424. The electrically insulative effect of the bubbles 460 increases the impedance and causes the phase angle to be more capacitive than when the electrode 424 was in contact with the target tissue 410. Accordingly, the fourth stage 400d corresponds with a relatively high measured impedance on the plot of impedance 302 and a relatively capacitive measured phase angle on the plot of phase angle 304.

In the case of the clinician employing a running start strategy—in which the energy signal is applied to the electrode within the delivery component and retracted from the tissue and then the energized electrode is physically advanced to contact and cross the target tissue—the phase angle is initially relatively capacitive due to the low mobility of ions within the delivery component, such as the dilator. Also, an electrically insulative gaseous layer can form around the heated electrode due to a saline flush within the delivery component. Thus, when the electrode is energized within the delivery component prior to contacting tissue, the impedance is relatively high and relatively capacitive as in the case after a crossing. When the energized electrode is advanced to contact the target tissue, however, the gaseous layer is displaced, and the phase angle becomes relatively resistive and the impedance magnitude drops.

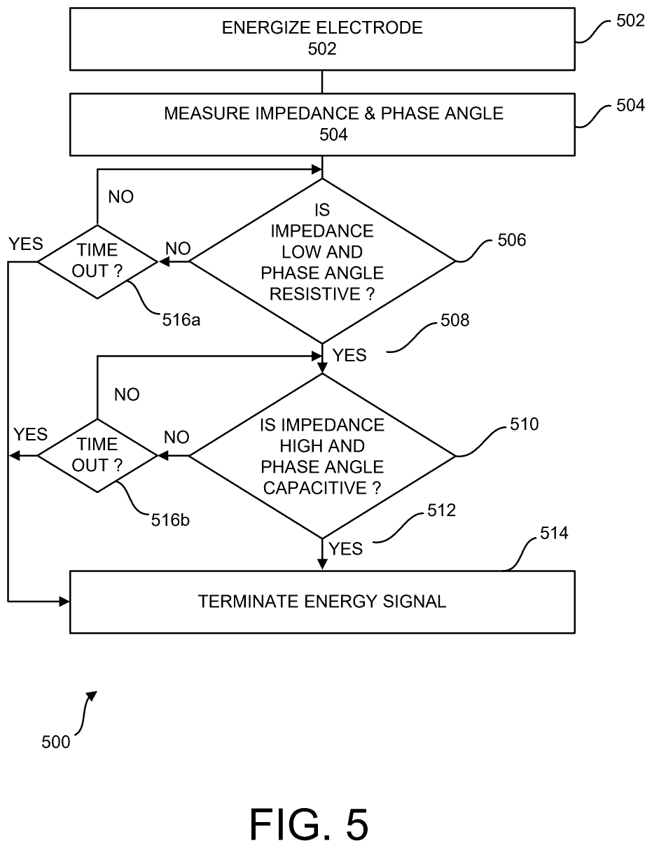

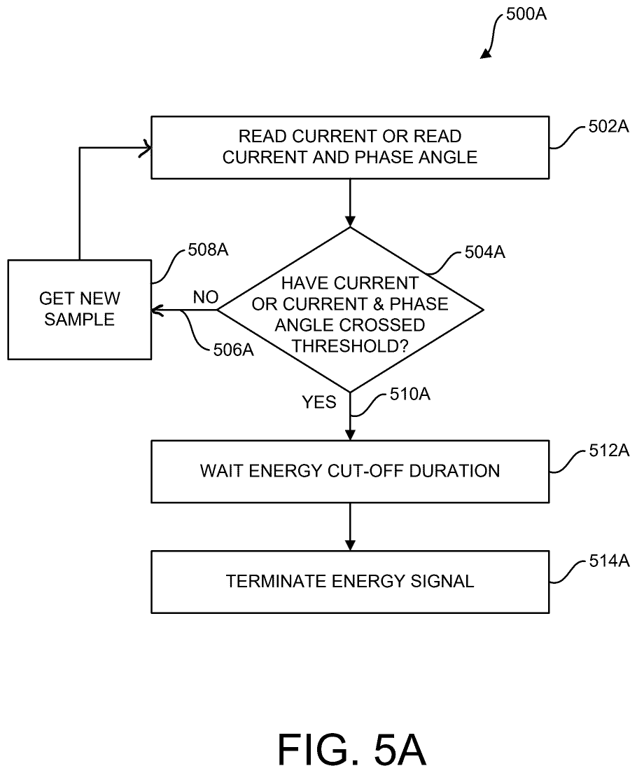

FIG. 5 illustrates an example method 500 that can be implemented in the controller, such as via a set of executable instructions, to automatically generate an indication of tissue crossing, such as terminate the energy signal or provide a notice like an audio signal to output speakers or display alert to a display device such as output device 107, once a tissue crossing is suspected. In the method, an energy signal is applied to the electrode at 502. The controller 212 measures the impedance of the electrode load at the electrode of the puncture device, the phase angle of the energy signal throughout the application of the energy signal, or both the impedance of the electrode load at the electrode of the puncture device and the phase angle of the energy signal throughout the application of the energy signal at 504. In some embodiments, the controller continuously measures, such as continuously samples, the signals to determine impedance and phase angle. The method 500 determines whether the impedance is relatively low and the phase angle is relatively resistive at 506, indicating the likelihood of the energized electrode in contact with tissue. In one embodiment of an implementation of method 500, preselected first threshold amounts are set or and applied against the measured impedance and phase angle to determine if the thresholds have been traversed and the impedance and phase angle have changed. For instance, a first impedance threshold can be set relatively low in the expected range of impedances of the system and a first or resistive phase angle threshold can be set relatively high in the expected range of phase angles of the system. In one embodiment, if the measured impedance is below the first threshold impedance, the measured phase angle is above the first threshold phase angle, or both the measured impedance is below the first threshold impedance and the measured phase angle is above the first threshold phase angle, the measured signals indicate a high likelihood that energized electrode is in contact with tissue.

If the impedance is relatively low, the phase angle is relatively resistive, or both at 508, the method 500 proceeds to determine whether the impedance is relatively high and the phase angle is relatively capacitive at 510, indicating the likelihood of that the energized electrode has crossed the tissue. In one embodiment of an implementation of method 500, preselected second threshold amounts can be set and applied against the measured impedance and phase to determine if the thresholds have been traversed and the impedance and phase angle have changed. For instance, a second impedance threshold can be set relatively high in the expected range of impedances of the system and a second phase angle threshold can be set relatively low in the expected range of phase angles of the system. If the measured impedance is above the second threshold impedance and the measured phase angle is below the second or capacitive threshold phase angle, the measured signals indicate a high likelihood that energized electrode has crossed tissue. In another embodiment, the method looks for a characteristic of the energy signal, such as a selected slope in the plot of the impedance and a selected slope in the plot of the phase angle rather than before and after amounts, such as a selected change in impedance during a selected time period to determine the likelihood that the energized electrode has crossed tissue at 512.

If the impedance is relatively high and the phase angle is relatively capacitive at 510, or the slope of the plots are such as to indicate the likelihood that the energized electrode has crossed the tissue, at 512, an indication of crossing is generated, such as the energy signal is terminated at 514.

In this embodiment, the controller 212 monitors the impedance and phase angle to determine whether the energized electrode is likely in contact with tissue at 506 to avoid a false positive that would terminate the energy signal if the activated electrode was still within the delivery component in a running start strategy. For example, the electrical characteristics of a running start, with the activated electrode within the delivery component, mimic that of an activated electrode in the left atrium. To reduce the chances for a false positive, the method 500 monitors the electrical characteristics at 504 and determines a likelihood that the electrode has been in contact with tissue at 508 before determining the likelihood that the electrode has crossed at 512 rather than simply terminating the energy signal if the impedance is high and the phase angle is capacitive.

If at step in the method 500, a time-based expiration has lapsed, such as at 516a, 516b, which can include cases where the energized electrode is left in the dilator too long, the puncture is signal is deactivated at 514.

In one embodiment, the controller 212 can set the thresholds or plot characteristics in the software 226 to implement the method 500. The thresholds or plot characteristics used to determine tissue contact and crossing likelihoods can be preselected and based on features of the system 100. In one embodiment, the controller 212 can load into memory 224 for use with program 226 parameters 111 stored on the memory device associated with the crossing device to determine ranges of possible impedances, ranges of phase angles, thresholds for impedances, thresholds for phase angles, or plot characteristics of impedances or phase angles for use in method 500. In one embodiment, a change in plot characteristic can include a change in slope of the measured electrical impedance overtime or the measured phase angle over time.

Impedance of the electrical load at the electrode can be determined in some embodiments according to Ohms law, which is voltage in volts is equal to current in amperes multiplied by resistance in Ohms. Distinctions between real power and apparent power can be made in the determinations. For example, apparent power is voltage in volts multiplied by current in amperes; and real power is voltage in volts multiplied by current in amperes multiplied by the cosine of the phase angle in radians. In one embodiment, current and voltage are measured via measurement circuit and provided to the controller. In another embodiment, the electrosurgical generator can be configured to control the voltage of the energy signal, so that variations in current can be used as an inferential measurement of impedance. For example, if voltage is held relatively constant, then a relatively higher current is inferential of a relatively lower impedance, and a relatively lower current is inferential of a relatively higher impedance. In still another embodiment, the voltage can be used as inferential measurement of impedance. For example, the application of a power limiter circuit in the generator 102 will pull down the voltage when a low impedance load is connected, and the release of the voltage indicates a higher impedance load is connected. Phase angle describes an amount of phase shift between total voltage and total current. Phase angle can be expressed as radians or degrees, such as from (0 to 360 degrees) or from (−180 to 180 degrees) and can represent the amount by which the voltage is either leading or lagging the current. In embodiments, the phase angle can be determined by software or phase angle measurement hardware.

To perform the embodiment of the method 500 in controller 212, the data used to determine impedance and phase angle is sampled at a relatively fast sampling rate. In one embodiment, a Fourier transform can be applied to determine a root-mean-square (RMS) magnitude of the voltage, current, or voltage and current of the energy signal with a simultaneous capture of the phase angle, such as via the measurement circuit 210. The Fourier transform can operate at frequencies approaching the frequency of the RF energy signal, which can provide an appropriate temporal resolution of data.

Occasionally, noise or effects during tissue vaporization, such as momentary bubble formation or an electrode retracted from tissue contact, can lead to impedance or phase angle measurements that cross set thresholds in a process to monitor impedance and phase angle of the energy signal. To account for such issue and avoid false positives that prematurely terminate the energy signal, the controller 212 can be configured to respond to continuous or stable readings rather than momentary threshold traversals. For example, the energy signal will only be terminated based on a sustained monitored change of electrical impedance or phase angle. In one embodiment, the sustained change can include a selected number of successive readings, such as successive readings past the preselected threshold. In another embodiment, the sustained change can include a select period of time of successive readings, such as successive readings past the preselected threshold. Such a period of time can be selected to be less than the pre-set time expiration for the energy signal. In other examples, sustained change can be determined via determining a rolling average of the readings or effected via lowpass filtering of the readings in hardware or software.

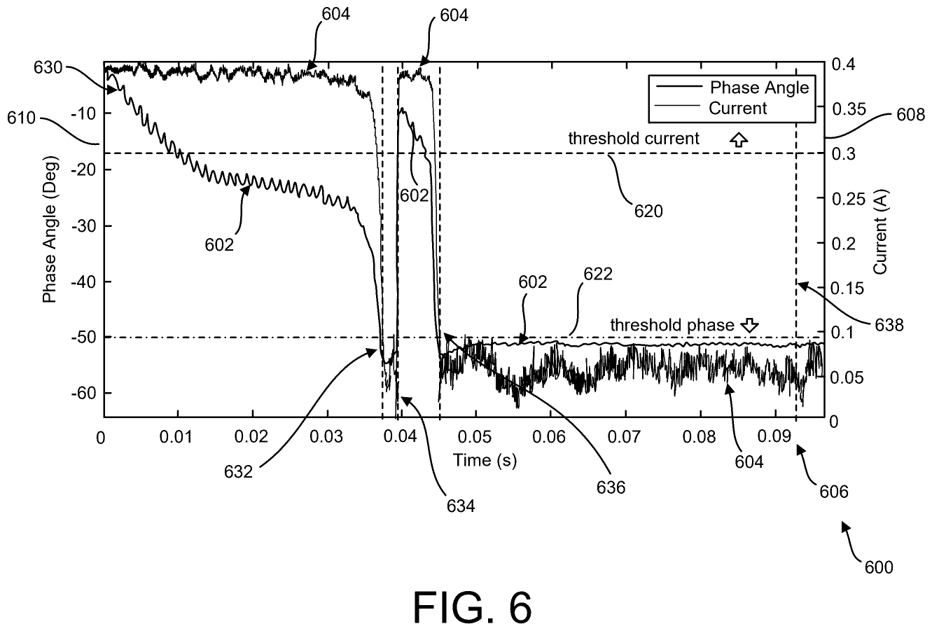

FIG. 6 illustrates a graph 600 including a plot of current magnitude 602 superimposed with a plot of phase angle 604 of energy signal as measured at the electrosurgical generator 200 with respect to time as a transseptal guidewire is applied to form a puncture in target tissue in an example procedure. A timeline, or time since activation of the energy signal in the example procedure in seconds 606 is presented along the x-axis. Current in amperes 608 is presented along the right-side y-axis. Phase angle in degrees 610 is presented along the left-side y-axis. Time 606 of the procedure ranges from zero seconds to approximately 0.1 seconds. Current 608 ranges from 0 amperes to 0.4 amperes on the right-side y-axis. Phase angle 610 on the graph along the left-side y-axis ranges from less than negative sixty degrees to zero degrees. For example, resistive phase angles are greater than negative forty-five degrees and capacitive phase angles are less than negative forty-five degrees.

In the example, the measured current is used as an analog, or an inferential measurement, of impedance because the radiofrequency generator is voltage controlled. Accordingly, a relatively high current is inferential of a relatively low impedance, and a relatively low current is inferential of a relatively high impedance.

For monitoring the electrical impedance and phase angle, such as via method 500, a current threshold is selected at 620, such as at 0.3 amperes, and a phase angle threshold is selected at 622, such as at negative fifty degrees.

As treatment starts at approximately 630 with respect to the timeline, the current plot 602 is above the current threshold 620 and the phase angle is relatively resistive, indicative of activated electrode against tissue. This can correspond with 508 of method 500.

The phase angle plot 604 drops below the phase angle threshold 622 at 632. In one embodiment, this can correspond with 512 of method 500. Rather than terminate the energy signal at 514 in this embodiment, the controller 212 begins to monitor for a sustained change. In one embodiment, the controller 212 begins a cut-off timer of approximately 0.05 seconds. If the phase angle plot 604 remains below the phase angle threshold 622 for the duration of the cut-off timer, in this case 0.05 seconds, the controller 212 will terminate the energy signal at 514.

At 634, less than 0.01 seconds after the phase angle plot 604 dropped below the threshold 622, the phase angle plot 604 exceeds the threshold 622. Accordingly, the phase angle measurement did not incur a sustained change at 634. The cut-off timer is stopped and reset. The controller 212 does not terminate the energy signal at 514.

The phase angle plot 604 drops below the phase angle threshold 622 at 636. At this time, the controller again begins to monitor for a sustained change. In one embodiment, the controller 212 begins a cut-off timer of approximately 0.05 seconds. The phase angle plot 604 remains under the phase angle threshold for the duration of the cut-off timer, and the controller 212 terminates the energy signal at 638.

FIG. 5A illustrates an example method 500A that can be implemented in the controller, such as via a set of executable instructions, to automatically generate an indication of tissue crossing, such as terminate the energy signal or provide a notice like an audio signal to output speakers or display alert to a display device such as output device 107, once a tissue crossing is suspected. Method 500A is an embodiment of method 500 of FIG. 5.

An energy signal is applied to the electrode, and the controller 212 measures the impedance of the electrode load at the electrode of the puncture device or an electrical characteristic that is used as an inferential measurement of impedance, or the controller 212 measures the impedance or the electrical characteristic used as an inferential measurement of impedance and the phase angle of the energy signal throughout the application of the energy signal at 502A. In some embodiments, the controller continuously measures, such as continuously samples, the signals to determine impedance or the inferential measurement of impedance or the controller continuously measures the signals to determine impedance or the inferential measurement of impedance and phase angle. In the illustrated example, the controller of a voltage controlled electrosurgical generator continuously samples electrical current as an inferential measurement of impedance during the application of the energy signal to the electrode or the controller continuously samples electrical current and phase angle during the application of the energy signal to the electrode.

The method 500A determines whether the energy signal, such as an electrical characteristic of the sample of the energy signal, has traversed a predefined threshold such as a threshold current, or predefined thresholds such as a threshold current value and a threshold phase angle at 504A. For example, a determination is made as to whether the measured electrical current sample has, or set of measured electrical current samples have, values that have fallen below the threshold current amount or the measured current sample and corresponding phase angle has fallen below the threshold current amount and fallen below the threshold phase angle. In order to reduce the likelihood of false positives, the determination can be made after a sustained change in the electrical characteristic with respect to the threshold. If the threshold is not traversed, at 506A, another sample or set of samples of the energy signal is received at 508A, and the impedance or the electrical characteristic used as an inferential measurement of impedance and the phase angle of the energy signal is read again at 502A. If the threshold is traversed, at 510A, the energy signal is terminated.

In the illustrated embodiment, the determination that the threshold has been traversed begins an energy cut-off timer, and once the energy cut-off timer has reached a predefined time limit at 512A, the energy signal is terminated at 514A. With the application of the energy cut-off timer, an energy cut-off delay can be implemented to allow the energy signal to continue a relatively short amount of time to complete any remaining puncture yet still avoid adverse effects of a prolonged energy signal applied to a patient. In one example, however, the predefined time limit of the energy cut-off timer can be zero seconds, such that the energy signal is terminated immediately after the threshold has been determined to be traversed (such as via a determined sustained change with respect to the threshold) and without an energy cut-off delay.

In one embodiment, the measurement of phase angle is used to verify the puncture for further confidence, and if the measured phase angle transitions from a resistive to capacitive across a predefined phase angle threshold and the impedance or inferential impedance transitions across the predefined impedance or inferential impedance threshold, the energy cut-off delay is started to terminate the energy signal.

In the embodiment in which the determination of whether a threshold has been traversed includes a determination of that the measured electrical current has fallen below a threshold value (or that the measured impedance has exceeded a threshold value) can include procedures in which the electrode is placed into contact with the tissue, such as used to tent the septum, prior to the application of an energy signal to the electrode to activate the electrode.

In some embodiments, the determination of whether a threshold has been traversed, such as whether a threshold current or impedance value has been traversed, includes a determination of whether the electrical current measured in the energy signal has exceeded the threshold current value. In such an embodiment, the electrical current profile in the energy signal progresses from a relatively low amount to a relatively high amount with respect to time during the tissue crossing procedure.

Such a profile is possible in a “running start” situation if the electrode is activated while inside of the delivery component, such as a dilator, and then moves the activated electrode to blood or tissue. In one example, the activated electrode may lack conductive fluid contact or may contact an exposed hypotube within the delivery component, which causes a relatively higher impedance and capacitive phase angle condition. Once the activated electrode exits the delivery component, one of at least the following situations occur. For some electrodes falling within a given ratio of energy to surface area to energy, a bubble can form while inside the dilator and adhere to the electrode even after exiting the tip. Once the electrode contacts tissue, this bubble displaces and causes the standard low impedance resistive delivery detect as puncture, and then the bubble reforms after crossing. If a device has a larger tip surface area, or is otherwise less stable than the example above, upon exit from the delivery component, the activated electrode exhibits a relatively high current, resistive delivery upon exit from the delivery component. This device may never stabilize back into the capacitive, high impedance type signal. After a certain time after this “running start”, a puncture in the tissue will occur. Method 500A applied with electrical current threshold values that are to be exceeded are detecting an exit from the dilator and then waiting a time, via a non-zero energy cut-off duration at 512A, to terminate.

In still another embodiment, the measurements of impedance and phase angle or the inferential measurement of impedance, such as current in a voltage controlled electrosurgical generator, and phase angle can be substituted for measurements of real power and apparent power. In this embodiment, the measurements of real power and apparent power are inferential measurements of impedance and phase angle. For instance, in a voltage controlled electrosurgical generator or an electrosurgical generator having a power limiter circuit to pull down voltage, electrical current and voltage are inferential measurements of impedance and are used to calculate real power and apparent power while phase angle is used to determine real power. A real power determination relatively similar to an apparent power determination of the energy signal is indicative of a resistive phase angle whereas a real power determination relatively dissimilar to the apparent power determination of the energy signal is indicative of a capacitive phase angle.

In one embodiment, real power and apparent power of the energy signal are determined and compared to each other. If the determined real power is within a predefined threshold divergence of the determined apparent power, such as within five percent or ten percent, the controller can determine a likelihood that the electrode is in contact with the septum. If, on the other hand, the determined real power is greater than a predefined threshold of divergence of the determined apparent power, such as greater than twenty percent, the controller can determine a likelihood that the electrode has crossed the septum and is located in the left atrium of the patient's heart.

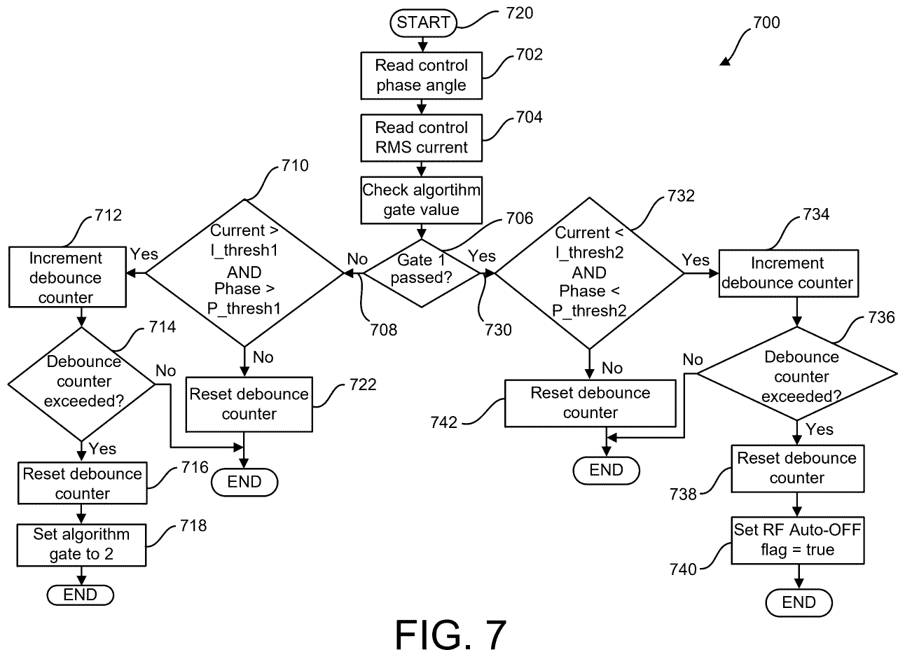

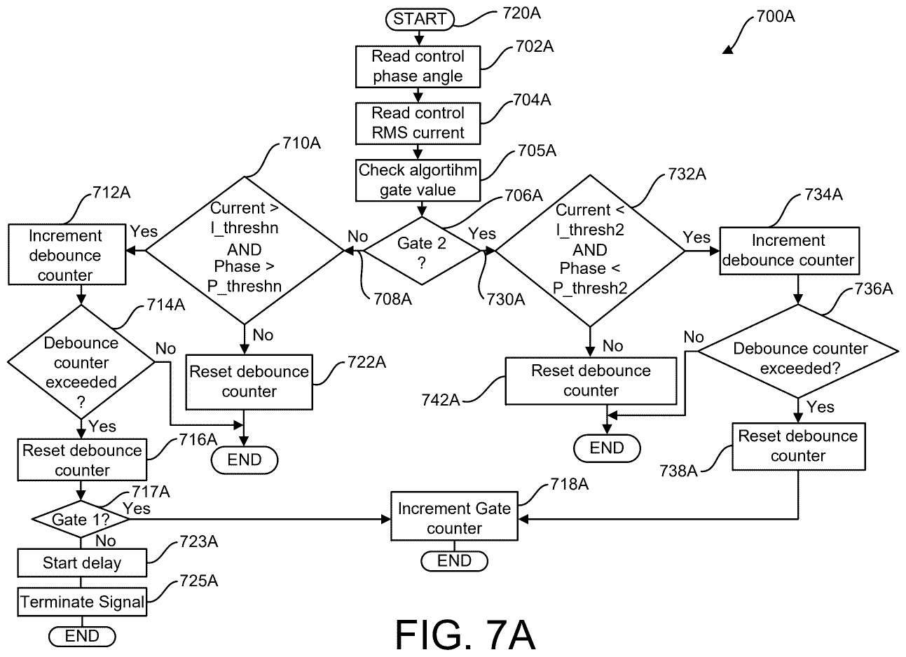

FIG. 7 illustrates a method 700 applied for each reading during monitoring of the impedance and phase angle that is an embodiment of method 500. Method 700 applies multiple thresholds to determine the likelihood that the electrode is in contact with tissue such as at 506 and to determine the likelihood that the electrode has crossed tissue at 510. Method 700 incorporates a determination of a sustained change within its process to avoid false positives prior to terminating the energy signal. In the embodiment of the method 700, monitored current is used as an inferential measure of impedance, such that a high current is inferential of a low impedance, and vice versa, because the radiofrequency generator is voltage controlled. Accordingly, for each reading, phase angle is monitored at 702 and RMS current is monitored at 704. (For circumstances in which RMS current or RMS values are monitored, those skilled in the art recognize that peak-to-peak or amplitude values can be substituted.) The method 700 can also check whether a first gate (gate 1), an indication that the electrode has been in sustained contact with tissue, has passed at 706.

In the embodiment of method 700, a counter is employed to determine if the likelihood of contact with tissue has been sustained and continuous. The counter is also employed to determine if the likelihood that the electrode has crossed has been sustained and continuous. Each reading either increments or resets the counter. Once the counter reaches or surpasses a preselected number of readings, the contact with tissue is determined to be sustained and continuous or the crossing is determined to be sustained and continuous. In this example, a counter of reading rather than a timer is used to determine a sustained change.

If the first gate has not been passed at 708, or that the activated electrode has not been in sustained contact with tissue, the controller determines whether the activated electrode is currently likely in contact with tissue, such as via comparison of the current reading and phase angle measurement to respective thresholds at 710. In the illustrated embodiment, the controller compares whether the current measurement has traversed a first current threshold and whether the phase angle measurement has traversed a first (resistive) phase angle threshold at 710. The method 700 increments the counter at 712 if the measurements traverse the thresholds at 710. If the counter has reached the preselected number of readings at 714, the controller resets the counter at 716 and sets a flag to indicate that gate 1 has passed, i.e., the controller sets a gate 1 flag, at 718. Accordingly, the controller has determined via the counter that the readings indicating a likelihood of the electrode in contact with tissue are sustained and continuous. The process 700 begins again at 720. If the current and phase angle measurements do not traverse the respective first thresholds at 710, the counter is reset at 722, and the process 700 begins again at 720.

Once the gate 1 flag is set at 730, the controller determines whether the activated electrode has likely crossed the tissue, such as via comparison of the current reading and phase angle measurement to respective thresholds at 732. In the illustrated embodiment, the controller compares whether the current reading has traversed a second current threshold and whether the phase angle has traversed a second (capacitive) phase angle threshold at 732. The method 700 increments the counter at 734 if the measurements traverse the thresholds at 732. If the counter has reached the preselected number of readings at 736, the controller resets the counter at 738 and sets a flag to terminate the energy signal at 740. Accordingly, the controller has determined via the counter that the readings indicating a crossing are sustained and continuous. If the current and phase angle measurements do not traverse the respective second thresholds at 732, the counter is reset at 742, and the process 700 begins again at 720, with the gate 1 flag still set.

FIG. 7A illustrates a method 700A applied for each reading, or sample, during monitoring of the impedance and phase angle that is an embodiment of method 500. Method 700A applies multiple thresholds to determine the likelihood that the electrode is in contact with tissue such as at 506 and to determine the likelihood that the electrode has crossed tissue at 510. Method 700A also incorporates a determination of a sustained change within its process to avoid false positives prior to terminating the energy signal as in method 700. In one example, an electrical current profile may begin with a relatively high value and then transition to a relatively low value before transitioning to a relatively high value again during the puncture procedure. Method 700A detects currents exceeding a first threshold, then falling below a second threshold, and then exceeding a third threshold. The status of the measured value can be referred to as a gate, such as a relatively high value of the electrical current at the beginning of the procedure can be assigned gate 1, the subsequent relatively lower value of the electrical current cam be assigned gate 2, and the subsequent relatively higher value of the electrical current can be assigned gate 3. Such a profile can be exhibited via relatively unstable electrodes, as identified above, in electrosurgical generators that pre-charge the active electrode, or in poorly flushed delivery components prior to application of the energy signal. Such conditions can cause the measured current to demonstrate a false peak of the profile while the activated electrode is in the delivery component as the control loop circuitry adjusts to impedance while the activated electrode is within the delivery component, such as within the dilator. The electrical current profile with respect to time can progress from a false peak of relatively higher current to a relatively lower current due to high impedance, and then to a relatively higher current because of the puncture. Some electrosurgical devices can return to a relatively higher impedance (relatively lower electrical current) after the puncture, but some electrosurgical devices, such as some unstable electrosurgical devices, may never return to a relatively higher impedance.

In the illustrated embodiment, as in the illustrated embodiment of method 700, a counter rather than a timer is used to determine a sustained change. In the embodiment of the method 700A, monitored current is used as an inferential measure of impedance, such that a high current is inferential of a low impedance, and vice versa, because the radiofrequency generator is voltage controlled. Accordingly, for each reading, phase angle is monitored at 702A and RMS current is monitored at 704A. One skilled in the art can readily modify the method 700A to account for other determinations, such as impedance, impedance, and phase angle, voltage, voltage and phase angle, or a divergence between real power and apparent power, as well as other comparisons of measurements to signal characteristics.

The method 700A checks as to which gate or stage of the algorithm is to be applied at 705A. For instance, a check as to whether a second gate (gate 2), an indication that the electrode is in sustained contact with tissue, has been set at 706A. In one embodiment, flags can be set to indicate the status such as a gate 1 flag, gate 2 flag, or gate 3 flag, that can be read at 705A and used in the determination at 706A.

If the status of the process is not at gate 2 (or is at gate 1 or at gate 3), or that the activated electrode is not in sustained contact with tissue, at 708A, the controller determines whether the activated electrode is currently likely in contact with tissue, such as via comparison of the electrical current reading and phase angle measurement to respective thresholds at 710A. In the illustrated embodiment, the controller compares whether the current measurement has traversed an electrical current threshold and whether the phase angle measurement has traversed a (resistive) phase angle threshold corresponding with the status of the gate (gate 1 or gate 3) at 710A. In the embodiment, the thresholds for gate 1 is different than the thresholds for gate 3. The method 700A increments the counter at 712A if the measurements traverse the thresholds at 710A.

If the counter has reached the preselected number of readings at 714A, the controller resets the counter at 716A, determines whether the status is gate 1 at 717A, and if so, increments the gate count at 718A to gate 2 (sets a flag to indicate a status of gate 2). Accordingly, the controller has determined via the counter that the readings indicating a likelihood of the electrode in contact with tissue are sustained and continuous. The process 700A begins again at 720A.

If the counter has reached the preselected number of readings at 714A, the controller resets the counter at 716A, determines whether the status is gate 1 at 717A, and if not at gate 1, the process 700A terminates the energy signal such as via starting an energy cut-off delay at 723A and terminating the energy signal at 725A once the energy cut-off delay has expired.

If the current and phase angle measurements do not traverse the respective first thresholds at 710A, the counter is reset at 722A, and the process 700A begins again at 720A.

Once the gate 2 flag is set at 730A, such as determined at 706A, the controller determines whether the activated electrode has likely crossed the tissue, such as via comparison of the current reading and phase angle measurement to respective thresholds at 732A. In the illustrated embodiment, the controller compares whether the current reading has traversed a second current threshold and whether the phase angle has traversed a second (capacitive) phase angle threshold at 732A. The method 700A increments the counter at 734A if the measurements traverse the thresholds at 732A. If the counter has reached the preselected number of readings at 736A, the controller resets the counter at 738A and increments the gate count at 718A to gate 3. Accordingly, the controller has determined via the counter that the readings indicating a crossing are sustained and continuous. The process 700A begins again at 720A.

If the current and phase angle measurements do not traverse the respective second thresholds at 732, the counter is reset at 742, and the process 700 begins again at 720, with the gate 2 flag still set.

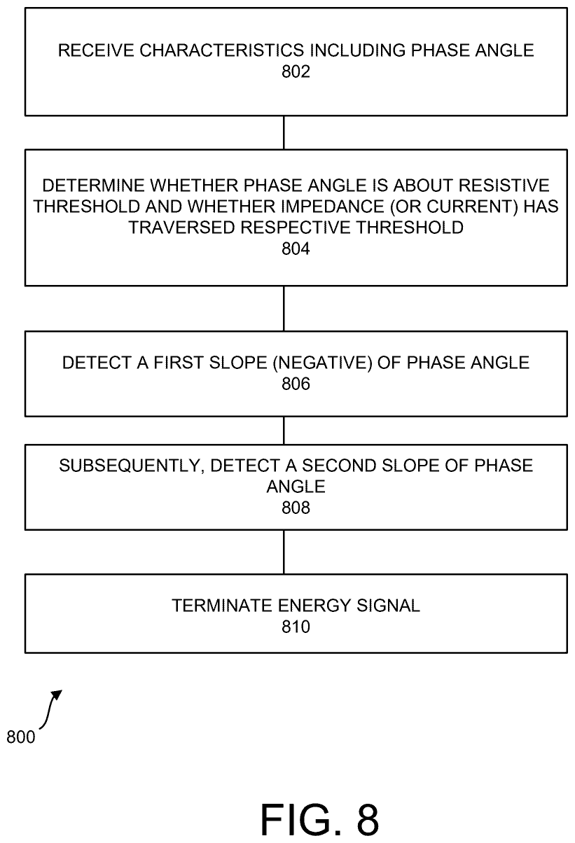

FIG. 8 illustrates a method 800 to monitor impedance and phase angle that is another embodiment of method 500. Method 800 monitors characteristics such as the amount of phase angle change over time, or phase angle slope, to make determinations as to the likelihood that tissue has been crossed to terminate the energy signal. Again, the controller can determine impedance or use current as an inferential measurement of impedance in method 800 as with method 700. In method 800, the controller receives measurements of current, phase angle, and phase angle slope at 802. Similar to 510 of method 500, the controller compares whether the current measurement has traversed a current threshold and whether the phase angle measurement has traversed a first (resistive) phase angle threshold at 804. This can indicate that the active electrode is in contact with tissue. If so, controller looks for a first slope, such as a negative slope, or drop in phase angle from a relatively resistive state to a relatively capacitive state over a first set period of time at 806. This can indicate that the active electrode has crossed the tissue. If so, the controller looks for a second slope, such as positive slop, or slight rise in phase angle in the relatively capacitive state over a second set period of time at 808. This can indicate a sustained change and eliminate false positives. If so, the controller terminates the energy signal at 810.

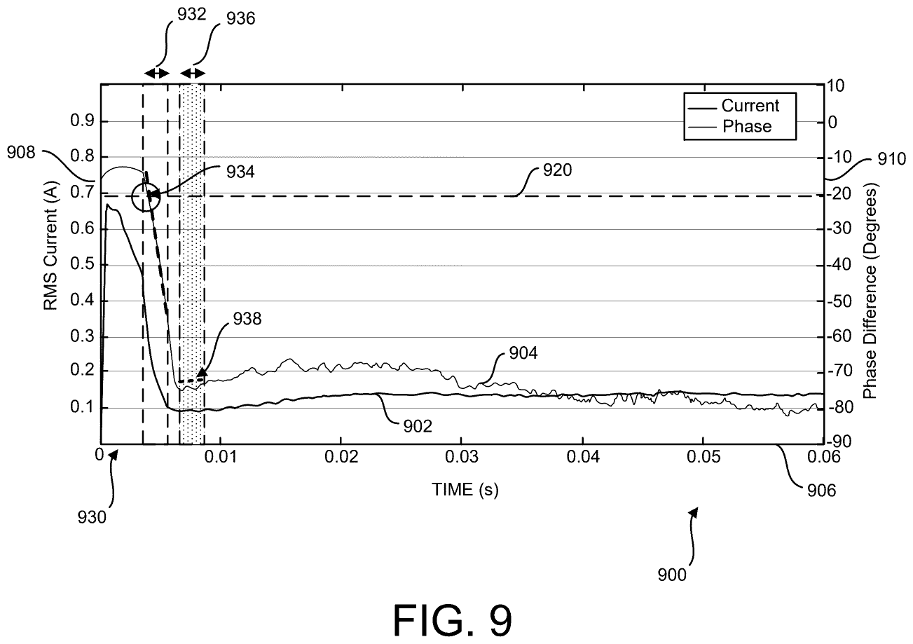

FIG. 9 illustrates a graph 900 including a plot of current magnitude 902 superimposed with a plot of phase angle 904 of energy signal as measured at the electrosurgical generator 200 with respect to time as a transseptal guidewire is applied to form a puncture in target tissue in an example procedure. In the example, the measured current is used as an analog, or an inferential measurement, of impedance because the radiofrequency generator is voltage controlled. A timeline, or time since activation of the energy signal in the example procedure in seconds 906 is presented along the x-axis. Current in amperes 908 is presented along the left-side y-axis. Phase angle in degrees 910 is presented along the right-side y-axis. An example phase angle resistive threshold 920 is set at approximately negative twenty degrees.

At approximately the beginning of treatment with respect to the timeline 906 at 930, the current plot is high, indicative of a low impedance, and phase angle has traversed a resistive threshold 920, which corresponds with 804 of method 800. During a first period of time 932, the slope of the phase angle plot 904 meets a set negative target 934, or selected slope, and changes from a relatively resistive phase angle measurement to relatively capacitive phase angle measurement, which corresponds with 806 of method 800. During a subsequent second period of time 936, the slope of the phase angle plot 904 meets a set positive target 938, or selected slope, and remains relatively capacitive, which corresponds with 808. Subsequently, the controller terminates the energy signal, which corresponds with 810.

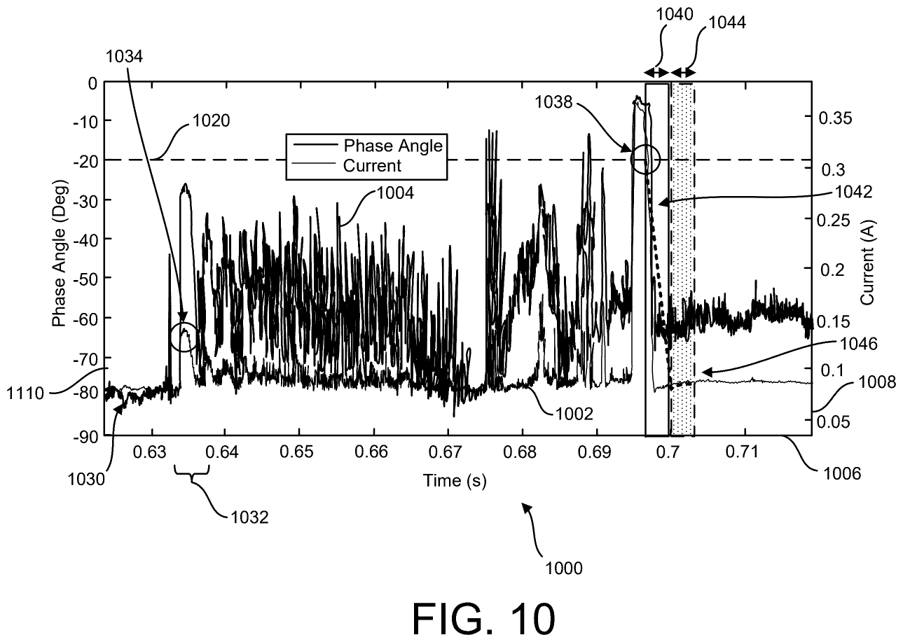

FIG. 10 illustrates a graph 1000 including a plot of current magnitude 1002 superimposed with a plot of phase angle 1004 of energy signal as measured at the electrosurgical generator 200 with respect to time as a transseptal guidewire is applied to form a puncture in target tissue in an example procedure. Graph 1000 illustrates current and phase measurements in a running start strategy and how method 800 accounts for false positives. In the example, the measured current is used as an analog, or an inferential measurement, of impedance because the radiofrequency generator is voltage controlled. A timeline, or time since activation of the energy signal in the example procedure in seconds 1006 is presented along the x-axis. Current in amperes 1008 is presented along the right-side y-axis. Phase angle in degrees 1010 is presented along the left-side y-axis. An example phase angle resistive threshold 1020 is set at approximately negative twenty degrees.

At approximately the beginning of treatment with respect to the timeline 1006 at 1030, the current plot is low, indicative of a high impedance, and phase angle is relatively capacitive, which corresponds with an electrode activated in the delivery component such as a dilator rather than in contact with tissue. During a first period of time 1032, the slope of the phase angle plot 1004 meets a set negative target 1034, but the phase angle measurement is not above the resistive threshold, so the slope is ignored. The phase angle traverses the resistive threshold at 1038. During a subsequent period of time 1040, the slope of the phase angle plot 1004 meets a set negative target 1042 and changes from a relatively resistive phase angle measurement to relatively capacitive phase angle measurement, which corresponds with 806 of method 800. During a still subsequent period of time 1044, the slope of the phase angle plot 1004 meets a set positive target 1046 and remains relatively capacitive, which corresponds with 808. Subsequently, the controller terminates the energy signal, which corresponds with 810.

In some embodiments, the controller 212 can facilitate presentation of a graph having a plot of current applied to the active terminal or electrical impedance magnitude of the electrical load at the device terminals superimposed on a plot of phase angle of the energy signal provided to an attached electrosurgical device 106 as a function of time on a display device such as included with output device 107 during a workflow, such as graphs 300, 600, 900, and 1000. In some embodiments, graphs 300, 600, 900 and 1000 are examples of visualizations on the output device 107. In some embodiments, the controller 212 can facilitate an output such as an audio alarm on speakers of display device once a puncture has been determined, such as once based on determined changes of electrical impedance and phase angle. In some embodiments, the controller 212 can facilitate presentation of indicators on the workflow of the time the energy signal was terminated.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. An electrosurgical system for tissue crossing, comprising:
a puncture device including an active electrode configured to deliver radiofrequency energy to tissue, wherein the active electrode has an electrode design that allows the active electrode to capture vapor generated by the active electrode;
an electrosurgical generator, electrically coupled to the active electrode, configured to generate a radiofrequency energy signal for delivery to the active electrode, the generator comprising:
a plurality of device terminals including an active terminal configured to couple to the active electrode and a return terminal;
a measurement circuit configured to measure a complex impedance between the active terminal and the return terminal, the complex impedance comprising an impedance magnitude and a phase angle; and
a controller operatively coupled to the measurement circuit and configured to:
monitor the impedance magnitude and the phase angle during delivery of a radiofrequency energy signal;
detect stable vapor bubble encapsulation of the active electrode based on the impedance magnitude increasing above a first threshold value and the phase angle becoming more capacitive than a second threshold value for a sustained period, wherein the sustained period indicates stable vapor bubble encapsulation representing successful tissue crossing; and
terminate the radiofrequency energy signal in response to determining that the sustained period has elapsed.

2. The system of claim 1, wherein the phase angle becoming more capacitive comprises the phase angle shifting from a first angle greater than negative forty-five degrees to a second angle less than negative forty-five degrees,
wherein the first angle indicates resistive electrical coupling through tissue and the second angle indicates capacitive electrical coupling through the vapor bubble encapsulation.

3. The system of claim 1, wherein detecting stable vapor bubble encapsulation comprises detecting that the impedance magnitude increases from a first impedance value between 200 Ohms and 800 Ohms during tissue contact to a second impedance value between 1200 Ohms and 3500 Ohms when the active electrode is positioned beyond the tissue in blood.

4. The system of claim 1, wherein the first threshold value is between 1200 Ohms and 3500 Ohms, and the second threshold value is between negative forty-five degrees and negative ninety degrees.

5. The system of claim 1, wherein the stable vapor bubble encapsulation corresponds to coalescence of multiple discrete vapor bubbles into a continuous electrically insulative gaseous layer encapsulating the active electrode.

6. The system of claim 1, wherein the controller is configured to:
detect a tissue contact condition based on the impedance magnitude being below a tissue contact threshold and the phase angle being greater than a resistive phase angle threshold, wherein the tissue contact condition indicates displacement of a gaseous layer from the active electrode by physical contact with the tissue; and
subsequent to detecting the tissue contact condition, detect the stable vapor bubble encapsulation based on the impedance magnitude increasing above the first threshold value and the phase angle becoming more capacitive than the second threshold value, wherein detection of stable vapor bubble encapsulation subsequent to the tissue contact condition represents successful tissue crossing.

7. The system of claim 1, wherein, to detect stable vapor bubble encapsulation for the sustained period, the controller is configured to:
initiate a cut-off timer when the impedance magnitude exceeds the first threshold value and the phase angle becomes more capacitive than the second threshold value; and
verify that the impedance magnitude and the phase angle continuously remain beyond their respective threshold values for a duration of the cut-off timer, wherein the duration is between 0.01 seconds and 0.1 seconds.

8. The system of claim 1, wherein, to detect stable vapor bubble encapsulation for the sustained period, the controller is configured to:
increment a counter for each successive measurement in which both the impedance magnitude exceeds the first threshold value and the phase angle is more capacitive than the second threshold value;
reset the counter when either the impedance magnitude falls below the first threshold value or the phase angle becomes less capacitive than the second threshold value; and
determine that stable vapor bubble encapsulation has occurred when the counter reaches a predetermined count value.

9. The system of claim 1, wherein, to detect stable vapor bubble encapsulation, the controller is configured to:
determine that a reactive power component exceeds a real power component by at least 20 percent, indicating capacitive energy delivery through an electrically insulative vapor layer.

10. The system of claim 1, wherein, to detect stable vapor bubble encapsulation, the controller is configured to:
detect a negative slope in the phase angle over time corresponding to vapor bubble formation and accumulation on the active electrode; and
detect a positive slope in the phase angle over time corresponding to stabilization of the vapor bubble encapsulation, wherein detection of the positive slope after the negative slope indicates formation of a stable vapor bubble layer.

11. The system of claim 1, wherein, to detect stable vapor bubble encapsulation for the sustained period, the controller is configured to:
determine a rolling average of successive measurements of the impedance magnitude and the phase angle, wherein the rolling average remaining beyond the respective threshold values for the sustained period confirms stable vapor bubble encapsulation.

12. The system of claim 1, wherein the controller is configured to apply lowpass filtering to measurements of the impedance magnitude and the phase angle in hardware or software to determine the sustained period, wherein lowpass filtered measurements remaining beyond the respective threshold values confirms stable vapor bubble encapsulation.

13. A method of detecting stable vapor bubble encapsulation during radiofrequency tissue crossing, the method comprising:
delivering radiofrequency energy via an active electrode to target tissue;
generating vapor bubbles on a surface of the active electrode as the active electrode is heated during delivery of the radiofrequency energy, wherein the vapor bubbles adhere to the surface of the active electrode;
measuring an impedance magnitude and a phase angle at the active electrode during delivery of the radiofrequency energy;
detecting formation of a stable vapor bubble layer encapsulating the active electrode based on the impedance magnitude increasing above a first threshold value and the phase angle shifting to a capacitive value below a second threshold value for a sustained period, wherein persistence of the stable vapor bubble layer for the sustained period indicates that the active electrode has crossed the target tissue and entered a liquid medium beyond the target tissue; and
automatically terminating delivery of the radiofrequency energy in response to determining that the stable vapor bubble layer has persisted for the sustained period.

14. The method of claim 13, wherein generating vapor bubbles comprises heating blood surrounding the active electrode to a temperature exceeding a boiling point of the blood, causing vaporization and nucleation of vapor bubbles on the surface of the active electrode.

15. The method of claim 13, comprising:
before detecting formation of the stable vapor bubble layer, detecting resistive electrical coupling through the target tissue based on the impedance magnitude being below a tissue impedance threshold and the phase angle being above a resistive phase angle threshold,
wherein physical contact between the active electrode and the target tissue displaces a pre-formed gaseous layer from the surface of the active electrode,
wherein detecting formation of the stable vapor bubble layer comprises detecting reformation of vapor bubbles after tissue crossing is complete by detecting that the impedance magnitude and the phase angle remain beyond their respective threshold values continuously for the sustained period.

16. The method of claim 13, wherein the impedance magnitude increases from a first value between 200 Ohms and 800 Ohms during tissue contact to a second value between 1200 Ohms and 3500 Ohms during stable vapor bubble encapsulation.

17. The method of claim 13, wherein determining that the stable vapor bubble layer persists for the sustained period comprises confirming that the impedance magnitude and the phase angle remain beyond their respective threshold values for a duration between 50 milliseconds and 100 milliseconds.

18. The method of claim 13, wherein the active electrode is constructed from a flexible shape memory material including a nickel titanium alloy or nitinol having an insulative outer coating,
wherein the radiofrequency energy is delivered as an alternating current having a frequency in a range of 100 kilohertz to 10 megahertz.

19. An electrosurgical tissue puncture system comprising:
a puncture device including:
an electrosurgical transseptal guidewire having a puncture wire shaft constructed from an electrically conductive material having an insulative outer coating, the puncture wire shaft including a puncture wire distal tip having a puncture electrode configured as an active electrode adapted to deliver radiofrequency energy, wherein the puncture electrode has an electrode design that allows the puncture electrode to capture vapor generated by the puncture electrode in blood; and
a delivery component including an elongated shaft defining a longitudinally extending axial lumen, wherein the electrosurgical transseptal guidewire is disposed within the axial lumen and the puncture electrode is extendable from a distal tip of the delivery component;
an electrosurgical generator electrically coupled to the puncture device, the electrosurgical generator comprising:
a plurality of device terminals including an active terminal electrically coupled to the puncture electrode and a return terminal;
a ground pad dispersive electrode electrically coupled to the return terminal;
a measurement circuit configured to measure an impedance magnitude between the active terminal and the return terminal and a phase angle of an energy signal applied to the active terminal; and
a controller configured to:
generate a radiofrequency energy signal for delivery to the puncture electrode;
monitor the impedance magnitude and the phase angle during delivery of the radiofrequency energy signal;
detect stable vapor bubble encapsulation of the puncture electrode based on the impedance magnitude increasing above a first threshold value and the phase angle becoming more capacitive than a second threshold value, wherein the stable vapor bubble encapsulation corresponds to bubbles forming on and adhering to the puncture electrode and coalescing to form an electrically insulative gaseous layer encapsulating the entire puncture electrode when the puncture electrode is immersed in blood after crossing tissue;
determine that the impedance magnitude and the phase angle remain beyond their respective threshold values for a sustained period indicating tissue crossing; and
terminate the radiofrequency energy signal based on the determination.

20. The system of claim 19, wherein the controller is configured to:
detect an initial tissue contact condition based on the phase angle being greater than negative forty-five degrees prior to detecting stable vapor bubble encapsulation, wherein the initial tissue contact condition indicates that a gaseous layer is displaced from the puncture electrode when the energized puncture electrode contacts the tissue; and
subsequent to detecting the initial tissue contact condition, detect the stable vapor bubble encapsulation when the phase angle becomes less than negative forty-five degrees, wherein the stable vapor bubble encapsulation indicates that a vapor layer has encapsulated the puncture electrode after the tissue has been vaporized and the puncture electrode has entered blood, and wherein the radiofrequency energy signal flows through alternate parasitic capacitive pathways when the vapor layer encapsulates the puncture electrode.
